(19) United States
(12) Patent Application Publication
(10) Pub. No.: US 2023/0363628 A1
(43) Pub. Date: Nov. 16, 2023

(54) WIRE PUNCTURE OF STRICTURE FOR PANCREATICOBILIARY ACCESS

(71) Applicant: Olympus Corporation, Tokyo (JP)

(72) Inventors: Gloria Yee, Wellesley, MA (US); Kenji Murakami, Hino-shi (JP)

(21) Appl. No.: 18/313,689

(22) Filed: May 8, 2023

Related U.S. Application Data
(60) Provisional application No. 63/364,438, filed on May 10, 2022.

(51) Int. Cl. A61B 1/018; A61B 1/00; A61B 18/14

(52) U.S. Cl. CPC A61B 1/018; A61B 1/000096; A61B 18/1492; A61B 2017/003

(57) ABSTRACT

Systems, devices, and methods for endoscopic access to a target region in a patient pancreaticobiliary system from an entry site of a stricture is disclosed. The stricture can be opened up using an radio-frequency (RF)-based approach by delivering RF energy to a working head of a steerable elongate instrument positioned at the entry site, or a mechanical puncture-based approach by applying force to a working head made of material with substantial stiffness. A machine learning (ML) model can be trained to determine a proper pancreaticobiliary access method for the patient, such as between the RF-based approach and the mechanical puncture-based approach. At least a distal portion of the steerable elongate instrument can be advanced into the pancreaticobiliary region to perform a diagnostic or therapeutic operation therein.

### CROSS REFERENCE TO RELATED APPLICATIONS

This application is related to commonly assigned U.S. Provisional Patent Application Ser. No. 63/364,438, entitled “WIRE PUNCTURE OF STRICTURE FOR PANCREATICOBILIARY ACCESS”, filed on May 10, 2022 (Attorney Docket No. 5409.614PRV), which is incorporated by reference in their entirety.

### FIELD OF THE DISCLOSURE

The present document relates generally to endoscopic systems, and more particularly to apparatus and methods for endoscopically accessing a pancreaticobiliary region of a patient to perform diagnostic or therapeutic operations therein.

### BACKGROUND

Endoscopes have been used in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices or biological matter collection devices for sampling or treating an anatomical region, and providing suction passageways for collecting fluids (e.g., saline or other preparations), among other procedures. Examples of such anatomical region can include gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Some endoscopes include a working channel through which an operator can perform suction, placement of diagnostic or therapeutic devices (e.g., a brush, a biopsy needle or forceps, a stent, a basket, or a balloon), or minimally invasive surgeries such as tissue sampling or removal of unwanted tissue (e.g., benign or malignant strictures) or foreign objects (e.g., calculi). Some endoscopes can be used with a laser or plasma system to deliver energy to an anatomical target (e.g., soft or hard tissue or calculi) to achieve desired treatment. For example, laser has been used in applications of tissue ablation, coagulation, vaporization, fragmentation, and lithotripsy to break down calculi in kidney, gallbladder, ureter, among other stone-forming regions, or to ablate large calculi into smaller fragments.

In conventional endoscopy, the distal portion of the endoscope can be configured for supporting and orienting a therapeutic device, such as with the use of an elevator. In some systems, two endoscopes can work together with a first endoscope guiding a second endoscope inserted therein with the aid of the elevator. Such systems can be helpful in guiding endoscopes to anatomic locations within the body that are difficult to reach. For example, some anatomic locations can only be accessed with an endoscope after insertion through a circuitous path.

Peroral cholangioscopy is a technique that permits direct endoscopic visualization, diagnosis, and treatment of various disorders of patient biliary and pancreatic ductal system using miniature endoscopes and catheters inserted through the accessory port of a duodenoscope. Peroral cholangioscopy can be performed by using a dedicated cholangioscope that is advanced through the accessory channel of a duodenoscope, as used in Endoscopic Retrograde Cholangio-Pancreatography (ERCP) procedures. ERCP is a technique that combines the use of endoscopy and fluoroscopy to diagnose and treat certain problems of the biliary or pancreatic ductal systems, including the liver, gallbladder, bile ducts, pancreas, or pancreatic duct. In ERCP, an cholangioscope (also referred to as an auxiliary scope, or a “daughter” scope) can be attached to and advanced through a working channel of a duodenoscope (also referred to as a main scope, or a “mother” scope). Typically, two separate endoscopists operate each of the “mother-daughter” scopes. Although biliary cannulation can be achieved directly with the tip of the cholangioscope, most endoscopists prefer cannulation over a guidewire. A tissue retrieval device can be inserted through the cholangioscope to retrieve biological matter (e.g., gallstones, bill duct stones, cancerous tissue) or to manage stricture or blockage in bile duct.

Peroral cholangioscopy can also be performed by inserting a small-diameter dedicated endoscope directly into the bile duct, such as in a Direct Per-Oral Cholangioscopy (DPOC) procedure. In DPOC, a slim endoscope (cholangioscope) can be inserted into patient mouth, pass through the upper GI tract, and enter into the common bile duct for visualization, diagnosis, and treatment of disorders of the biliary and pancreatic ductal systems.

Biliary stricture occurs when a portion of the bile duct abnormally gets smaller or narrower, which may be caused by damage (e.g., surgery) to the bile duct, passage of gallstones to the bile duct, infections of the bile duct, pancreatitis, or cancer in the bile duct or pancreas. Endoscopic stricture management generally involves placing a stricture management device to open or dilate the narrowed or obstructed portion of the duct. Devices used for pancreaticobiliary stricture management include, for example, dilating catheters, balloon dilators, and stents. Dilating catheters are tapered cylindrical tubes with a central channel, and may be passed over a guidewire through the accessory channel of the side-viewing duodenoscope. Wire-guided balloon dilators are used in the bile duct and the pancreatic duct, inflated with dilute contrast to facilitate visualization during ERCP. Pancreaticobiliary stents are self-expandable devices that can be passed through a working channel of an endoscope and inserted into the obstructed bile duct to open the stricture in ERCP.

### SUMMARY

Endoscopic placement of stricture management devices in an obstructed or narrowed portion the pancreaticobiliary ductal system can be a complicated procedure. Conventionally, biliary endoscopic sphincterotomy is a prerequisite for placement of stricture management devices, calculi removal, tissue acquisition (e.g., biopsy), among other biliary interventions. Biliary endoscopic sphincterotomy (EST) generally refers to the cutting of the biliary sphincter and intraduodenal segment of the common bile duct following selective cannulation, using a high frequency current applied with a special knife, sphincterotome, inserted into the papilla. Biliary endoscopic sphincterotomy is either used solely for the treatment of diseases of the papilla of Vater, such as sphincter of Oddi dysfunction, or to facilitate subsequent therapeutic biliary interventions.

The operating physician's experience and dexterity play an important role in determining the success rate and patient outcome of endoscopic biliary procedures. Occasionally, biliary endoscopic sphincterotomy may be hampered by altered surgical anatomy or invasive tumors. With the duodenoscope designed to be stable in the duodenum, it can be more difficult to reach the duodenal papilla in surgically altered anatomy. Manipulation of the sphincterotome to achieve desired cutting can also be technically difficult in patients with altered anatomy of pancreaticobiliary system (e.g., the ampulla). Conventional endoscopic systems generally lack the capability of automated navigation guidance based on patient's unique anatomy. The endoscopic stricture management devices and techniques can be less effective for patients with altered surgical anatomy or invasive tumors, particularly when the operator has less experience with such devices and techniques.

The present disclosure describes alternative apparatus, devices, and methods for endoscopically accessing a target region in patient pancreaticobiliary system via an entry site at or around a stricture. According to one embodiment, a pancreaticobiliary access method comprises steps including navigating a steerable elongate instrument through a body cavity or channel toward a stricture adjacent to a target pancreaticobiliary region, delivering radio-frequency (RF) energy to an entry site of the stricture via a working head of the steerable elongate instrument to produce an opening sized to the pancreaticobiliary region. The entry site can be identified by applying an image of the stricture to a trained machine-learning (ML) model. At least a distal portion of the steerable elongate instrument can then be passed through the produced opening into the pancreaticobiliary region to perform a diagnostic or therapeutic operation therein.

According to another aspect of the present disclosure, a pancreaticobiliary access method comprises steps including navigating a steerable elongate instrument through a body cavity or channel toward a stricture adjacent to a target pancreaticobiliary region, positioning the working head of the steerable elongate instrument at an entry site of the stricture and applying a mechanical force thereto to produce an opening to the pancreaticobiliary region. The working head can be configured to achieve a higher amount of stiffness than other portions (e.g., the proximal portion) of the steerable elongate instrument as the working head approaches the stricture. The entry site can be identified by applying an image of the trained machine-learning (ML) model. At least a distal portion of the steerable elongate instrument can then be passed through the produced opening into the pancreaticobiliary region to perform a diagnostic or therapeutic operation therein.

According to another aspect of the present disclosure, an artificial intelligence (AI)-based decision system can select an appropriate pancreaticobiliary access approach for a patient, such as between an RF-based approach and a mechanical puncture-based approach, as described above catheter having a rigidized working head. A machine-learning (ML) model can be trained to identify an entry site at or around the stricture, and to determine a proper pancreaticobiliary access method for the patient, such as between the RF-based approach and the mechanical puncture-based approach.

Example 1 is a method for endoscopically accessing a pancreaticobiliary region of a patient, the method comprising: navigating a steerable elongate instrument through a body cavity or channel toward a stricture adjacent to the pancreaticobiliary region; delivering radio-frequency (RF) energy to an entry site of the stricture via a working head of the steerable elongate instrument to produce an opening to the pancreaticobiliary region; and passing at least a distal portion of the steerable elongate instrument through the produced opening into the pancreaticobiliary region to perform a diagnostic or therapeutic operation therein.

In Example 2, the subject matter of Example 1 optionally includes applying an image of the stricture to a trained machine-learning (ML) model to identify the entry site of the stricture.

In Example 3, the subject matter of any one or more of Examples 1-2 optionally includes the RF energy that can be applied to a stricture beside an ampulla of Vater to produce an opening to a common bile duct.

In Example 4, the subject matter of any one or more of Examples 1-3 optionally includes delivering the RF energy through an uncoiled wire portion on the working head of the steerable elongate instrument, the uncoiled wire portion electrically coupled to an RF power generator.

In Example 5, the subject matter of any one or more of Examples 1-4 optionally includes adjusting an RF energy delivered to the entry site of the stricture based at least on a characteristic of the stricture.

Example 6 is a method for accessing a pancreaticobiliary region of a patient, the method comprising: navigating a steerable elongate instrument through a body cavity or channel toward to a stricture adjacent to the pancreaticobiliary region, the steerable elongate instrument extended between a proximal portion and a distal portion, the distal portion including a working head configured to achieve a higher amount of stiffness than the proximal portion of the steerable elongate instrument as the working head approaches the stricture; positioning the working head of the steerable elongate instrument at an entry site of the stricture and applying a mechanical force thereto to produce an opening to the pancreaticobiliary region; and passing at least the distal portion of the steerable elongate instrument through the produced opening into the pancreaticobiliary region to perform diagnostic or therapeutic operation therein.

In Example 7, the subject matter of Example 6 optionally includes applying an image of the stricture to a trained machine-learning (ML) model to identify the entry site of the stricture.

In Example 8, the subject matter of any one or more of Examples 6-7 optionally includes the working head that can be made of material through a rigidization process.

In Example 9, the subject matter of any one or more of Examples 6-8 optionally includes the distal portion of the steerable elongate instrument that can be configured to have axially variable stiffness.

In Example 10, the subject matter of any one or more of Examples 6-9 optionally includes the steerable elongate instrument having the distal portion comprising struts spatially arranged to provide variable stiffness as the steerable elongate instrument changes its posture, including an increase in stiffness in response to a change from a bending posture to a straightening posture.

Example 11 is an endoscopic system, comprising: a steerable elongate instrument extended between a proximal portion and a distal portion, the distal portion including a working head configured to achieve a higher amount stiffness than other portions of the steerable elongate instrument as the working head approaches a stricture adjacent to a pancreaticobiliary region; and a controller configured to provide a control signal to an actuator to robotically facilitate navigation and manipulation of the steerable elongate instrument, including, via the actuator: position the working head of the steerable elongate instrument at an entry site of the stricture and apply a mechanical force thereto to produce an opening to the pancreaticobiliary region; and pass at least the distal portion of the steerable elongate instrument through the produced opening into the pancreaticobiliary region to perform a diagnostic or therapeutic operation therein.

In Example 12, the subject matter of Example 11 optionally includes a robot arm configured to detachably engage the steerable elongate instrument, and to automatically adjust position or navigation of the steerable elongate instrument via the actuator in response to the control signal.

In Example 13, the subject matter of any one or more of Examples 11-12 optionally includes the steerable elongate instrument that can be configured to be robotically positioned and navigated to a duodenal papilla or a portion of pancreaticobiliary system.

In Example 14, the subject matter of any one or more of Examples 11-13 optionally includes the working head that can be made of material through a rigidization process.

Example 15 is an endoscopic system, comprising: a steerable elongate instrument configured to be positioned and navigated in a patient anatomy; a controller configured to: receive an image of a stricture adjacent to a pancreaticobiliary region; and apply the received image of the stricture to at least one trained machine-learning (ML) model to identify an entry site of the stricture, and to determine a pancreaticobiliary access approach, between (i) an radio frequency (RF)-based approach and (ii) a mechanical puncture-based approach, to access the pancreaticobiliary region; and an output unit configured to provide the determined pancreaticobiliary access approach to a user.

In Example 16, the subject matter of Example 15 optionally includes the controller that can be further configured to: construct a training dataset comprising stored procedure data obtained from past endoscopic stricture management procedures on a plurality of patients using respective pancreaticobiliary access approaches including the RF-based approach or the mechanical puncture-based approach, the stored procedure data including (i) images of strictures of the plurality of patients and (ii) assessments of the pancreaticobiliary access approaches of the respective procedures; and train the ML model using the training dataset.

In Example 17, the subject matter of any one or more of Examples 15-16 optionally includes the steerable elongate instrument that can include a catheter, a guide wire, or a guide sheath including a lumen to pass a stricture management device therethrough.

In Example 18, the subject matter of any one or more of Examples 15-17 optionally includes the steerable elongate instrument that can include an endoscope, the endoscope including an imaging sensor to generate the image of the stricture.

In Example 19, the subject matter of any one or more of Examples 15-18 optionally includes the steerable elongate instrument that can be extended between a proximal portion and a distal portion, the distal portion including a working head having a higher amount of stiffness than other portions of the steerable elongate instrument, wherein the working head is configured to, in response to a puncture force applied thereto, puncture the entry site of the stricture to produce an opening sized to pass at least the distal portion of the steerable elongate instrument therethrough.

In Example 20, the subject matter of any one or more of Examples 15-19 optionally includes the steerable elongate instrument that can include, at a distal portion thereof, a working head configured to be electrically coupled to an RF power generator and to deliver RF energy to the entry site of the stricture to produce an opening sized to pass at least the distal portion of the steerable elongate instrument therethrough.

The systems, devices, and methods described herein can improve pancreaticobiliary access particularly in patients with surgically altered or complicated anatomy or malignant biliary strictures. Compared to conventional endoscopic pancreaticobiliary access approach (e.g., sphincterotomy), the RF-based or mechanical puncture-based approach as described herein are more controllable and easier to operate, can reduce procedure complexity and shorten procedure time. The AI-based pancreaticobiliary access decision system can help avoid or reduce risks and complications associated with direct cutting (e.g., sphincterotomy). Overall ERCP procedure success rate can be improved, and the healthcare cost associated with complications and procedure failures can be reduced.

The presented techniques are described in terms of health-related procedures, but are not so limited. This summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWING

FIGS. 1-2 are schematic diagrams illustrating an example of an endoscopy system for use in endoscopic procedures such as an ERCP procedure.

FIGS. 3A-3B are diagrams illustrating an example of peroral cholangioscopy involving direct insertion of a cholangioscope into patient bile duct as in a DPOC procedure, and a portion of patient anatomy where the procedure is performed.

FIG. 4 is a diagram illustrating an example of mother-daughter endoscopes used in an ERCP procedure, and a portion of patient anatomy where the procedure is performed.

FIG. 5A illustrates an example of a mechanical puncture-based approach for accessing a pancreaticobiliary region in a ERCP procedure.

FIG. 5B illustrates an example of an RF-based approach for accessing a pancreaticobiliary region in a ERCP procedure.

FIG. 6 is a flow chart illustrating an example method for accessing a pancreaticobiliary region to perform diagnostic or therapeutic operations therein.

FIG. 7 is a block diagram illustrating an example machine upon which any one or more of the techniques (e.g., methodologies) discussed herein may perform.

### DETAILED DESCRIPTION

This document describes systems, devices, and methods for endoscopic access to a target region in a patient pancreaticobiliary system from an entry site of a stricture. The stricture can be opened up using an radio-frequency (RF)-based approach by delivering RF energy to a working head of a steerable elongate instrument positioned at the entry site, or a mechanical puncture-based approach by applying force to a working head made of material with substantial stiffness. A machine learning (ML) model can be trained to determine a proper pancreaticobiliary access method for the patient, such as between the RF-based approach and the mechanical puncture-based approach. At least a distal portion of the steerable elongate instrument can be advanced into the pancreaticobiliary region to perform a diagnostic or therapeutic operation therein.

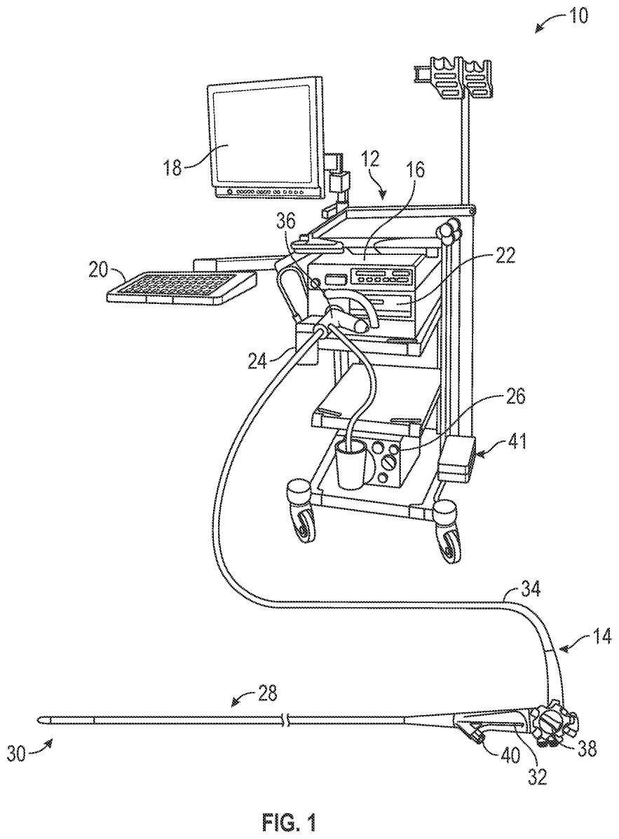

FIG. 1 is a schematic diagram illustrating an example of an endoscopy system 10 for use in endoscopic procedures, such as an ERCP procedure. The system 10 comprises an imaging and control system 12 and an endoscope 14. The endoscopy system 10 is an illustrative example of an endoscopy system suitable for patient diagnosis and/or treatment using the systems, devices and methods described herein, such as tethered and optically enhanced biological matter and tissue collection, retrieval and storage devices and biopsy instruments that can be used for obtaining samples of tissue or other biological matter to be removed from a patient for analysis or treatment of the patient. According to some examples, the endoscope 14 can be insertable into an anatomical region for imaging and/or to provide passage of or attachment to (e.g., via tethering) one or more sampling devices for biopsies, or one or more therapeutic devices for treatment of a disease state associated with the anatomical region.

The imaging and control system 12 can comprise a control unit 16, an output unit 18, an input unit 20, a light source 22, a fluid source 24, and a suction pump 26. The imaging and control system 12 can include various ports for coupling with endoscopy system 10. For example, the control unit 16 can include a data input/output port for receiving data from and communicating data to the endoscope 14. The light source 22 can include an output port for transmitting light to the endoscope 14, such as via a fiber optic link. The fluid source 24 can comprise one or more sources of air, saline or other fluids, as well as associated fluid pathways (e.g., air channels, irrigation channels, suction channels) and connectors (barb fittings, fluid seals, valves and the like). The fluid source 24 can be in communication with the control unit 16, and can transmit one or more sources of air or fluids to the endoscope 14 via a port. The fluid source 24 can comprise a pump and a tank of fluid or can be connected to an external tank, vessel or storage unit. The suction pump 26 can comprise a port used to draw a vacuum from the endoscope 14 to generate suction, such as for withdrawing fluid from the anatomical region into which the endoscope 14 is inserted.

The output unit 18 and the input unit 20 can be used by a human operator and/or a robotic operator of endoscopy system 10 to control functions of endoscopy system 10 and view output of endoscope 14. In some examples, the control unit 16 can additionally be used to generate signals or other outputs for treating the anatomical region into which the endoscope 14 is inserted. Examples of such signals or outputs can include electrical output, acoustic output, a radio-frequency energy output, a fluid output and the like for treating the anatomical region with, for example, cauterizing, cutting, freezing and the like.

The endoscope 14 can interface with and connect to the imaging and control system 12 via a coupler section 36. In the illustrated example, the endoscope 14 comprises a duodenoscope that may be use in a ERCP procedure, though other types of endoscopes can be used with the features and teachings of the present disclosure. The endoscope 14 can comprise an insertion section 28, a functional section 30, and a handle section 32, which can be coupled to a cable section 34 and the coupler section 36.

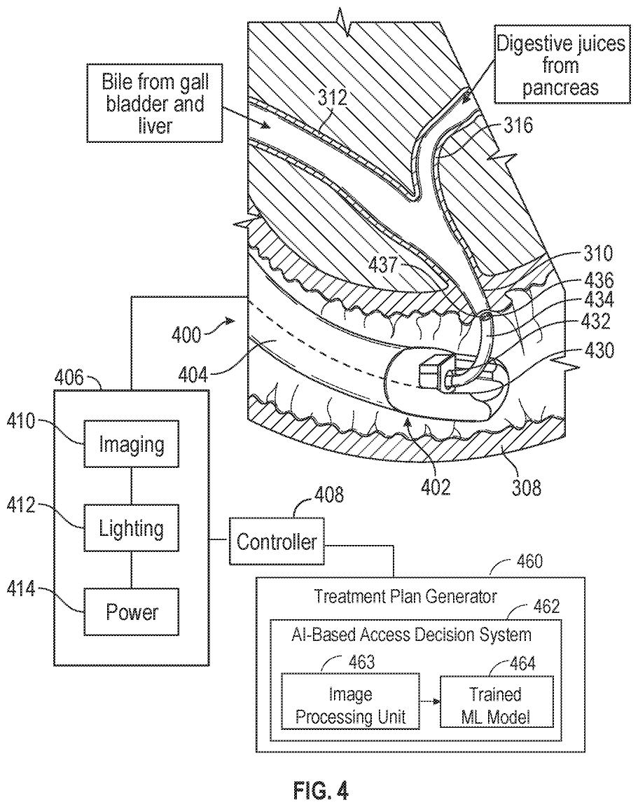

The insertion section 28 can extend distally from the handle section 32, and the cable section 34 can extend proximally from the handle section 32. The insertion section 28 can be elongate and include a bending section, and a distal end to which functional section 30 can be attached. The bending section can be controllable (e.g., by control knob 38 on the handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as a cholangioscope as shown in FIG. 4. The working channel can extend between handle section 32 and functional section 30. Additional functionalities, such as fluid passages, guidewires, and pull wires can also be provided by insertion section 28 (e.g., via suction or irrigation passageways, and the like).

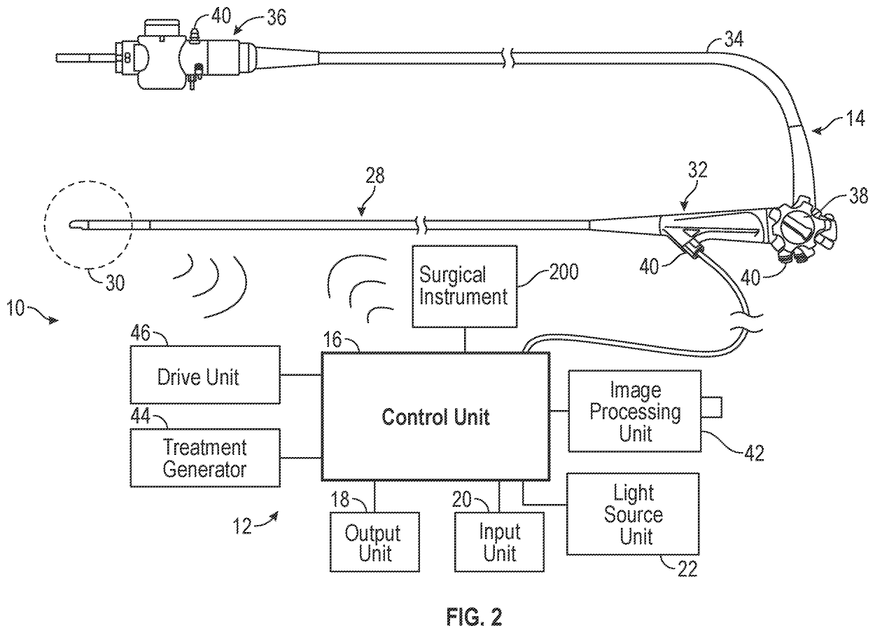

The handle section 32 can comprise a control knob 38 and ports 40. The ports 40 can be configured to couple various electrical cables, guidewires, auxiliary scopes, tissue collection devices of the present disclosure, fluid tubes and the like to handle section 32 for coupling with insertion section 28. The control knob 38 can be coupled to a pull wire, or other actuation mechanisms, extending through insertion section 28. The control knob 38 can be used by a user to manually advance or retreat the insertion section 28 of the endoscope 14, and to adjust bending of a bending section at the distal end of the insertion section 28. In some examples, an optional drive unit 46 (FIG. 2) can be used to provide motorized drive for advancing a distal section of endoscope 14 under the control of the control unit 16.

The imaging and control system 12, according to examples, can be provided on a mobile platform (e.g., cart 41) with shelves for housing light source 22, suction pump 26, image processing unit 42 (FIG. 2), etc. Alternatively, several components of the imaging and control system 12 shown in FIGS. 1 and 2 can be provided directly on the endoscope 14 such that the endoscope is “self-contained.”

The functional section 30 can comprise components for treating and diagnosing anatomy of a patient. The functional section 30 can comprise an imaging device, an illumination device, and an elevator. The functional section 30 can further comprise optically enhanced biological matter and tissue collection and retrieval devices. For example, the functional section 30 can comprise one or more electrodes conductively connected to handle section 32 and functionally connected to the imaging and control system 12 to analyze biological matter in contact with the electrodes based on comparative biological data stored in the imaging and control system 12. In other examples, the functional section 30 can directly incorporate tissue collectors.

In some examples, the endoscope 14 can be robotically controlled, such as by a robot arm attached thereto. The robot arm can automatically, or semi-automatically (e.g., with certain user manual control or commands), via an actuator, position and navigate an instrument such as the endoscope 14 (e.g., the functional section 30 and/or the insertion section 28) of in the target anatomy, or position a device at a desired location with desired posture to facilitate an operation of an anatomical target, such as a stricture management device to open or dilate an obstructed or narrowed portion of the ductal system. In various embodiments, a controller can use artificial intelligence (AI) to determine cannulation and navigation parameters and/or tool operational parameters (e.g., position, angle, posture, force, and navigation path), and generate a control signal to the actuator of the robot arm to facilitate operation of such instrument or tools in accordance with the determined navigation and operational parameters in a robotically assisted procedure.

FIG. 2 is a schematic diagram of the endoscopy system 10 shown in FIG. 1, which comprises the imaging and control system 12 and the endoscope 14. FIG. 2 schematically illustrates components of the imaging and control system 12 coupled to the endoscope 14, which in the illustrated example comprises a duodenoscope. The imaging and control system 12 can comprise a control unit 16, which can include or be coupled to an image processing unit 42, a treatment generator 44, and a drive unit 46, as well as the light source 22, the input unit 20, and the output unit 18 as discussed above with reference to FIG. 1. The control unit 16 can comprise, or can be in communication with, a surgical instrument 200 comprising a device configured to engage tissue and collect and store a portion of that tissue and through which an imaging device (e.g., a camera) can view target tissue via inclusion of optically enhanced materials and components. The control unit 16 can be configured to activate an imaging device (e.g., a camera) at the functional section of the endoscope 14 to view target tissue distal of surgical instrument 200 and endoscopy system 10, which can be fabricated of a translucent material to minimize the impacts of the camera being obstructed or partially obstructed by the tissue retrieval device. Likewise, the control unit 16 can be configured to activate the light source 22 to shine light on the surgical instrument 200, which can include select components that are configured to reflect light in a particular manner, such as tissue cutters being enhanced with reflective particles.

The image processing unit 42 and the light source 22 can each interface with the endoscope 14 (e.g., at the functional section 30) by wired or wireless electrical connections. The imaging and control system 12 can accordingly illuminate an anatomical region using the light source 22, collect signals representing the anatomical region, process signals representing the anatomical region using the image processing unit 42, and display images representing the anatomical region on the output unit 18. The imaging and control system 12 can include the light source 22 to illuminate the anatomical region using light of desired spectrum (e.g., broadband white light, narrow-band imaging using preferred electromagnetic wavelengths, and the like). The imaging and control system 12 can connect (e.g., via an endoscope connector) to the endoscope 14 for signal transmission (e.g., light output from light source, video signals from the imaging device such as positioned at the distal portion of the endoscope 14, diagnostic and sensor signals from a diagnostic device, and the like).

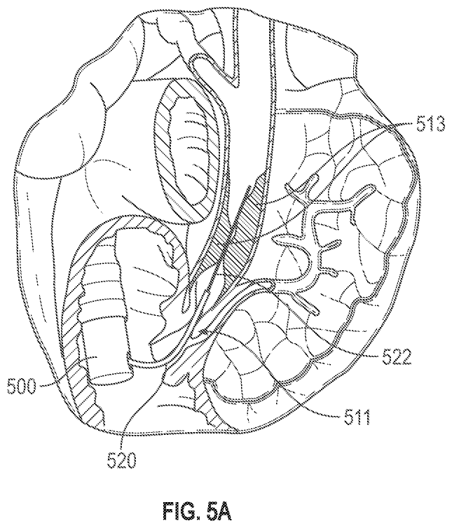

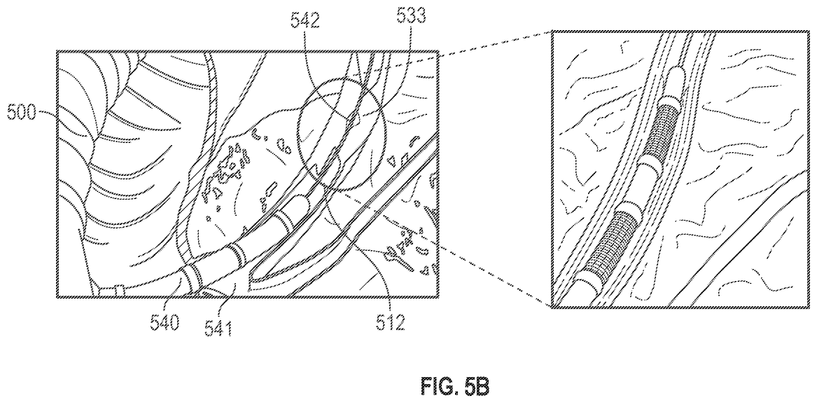

The treatment generator 44 can generate a treatment plan, which can be used by the control unit 16 to control the operation of the endoscope 14, or to provide with the operating physician a guidance for maneuvering the endoscope 14, during an endoscopic procedure. The treatment plan can include a pancreaticobiliary access plan for passing at least a portion of the endoscope 14 (or other steerable elongate instrument such as a catheter or a guidewire) into a target region of the pancreaticobiliary system to perform a diagnostic or therapeutic operation therein. Pancreaticobiliary access can be achieved by delivering radio-frequency (RF) energy to the stricture via a working head of the endoscope 14, or by applying mechanical force to the stricture via a working head of sufficient stiffness at a distal portion of the endoscope 14 (or other steerable elongate instrument such as a catheter or a guidewire). In an example, the treatment generator 44 can use a trained machine-learning (ML) model to determine patient candidacy for RF-based pancreaticobiliary access approach such as based on an endoscopic image of the stricture. For non-candidates, mechanical puncture-based pancreaticobiliary access may be recommended to an operating physician. Examples of the RF-based or mechanical puncture-based approaches for pancreaticobiliary access are discussed below with reference to FIGS. 5A-5B.

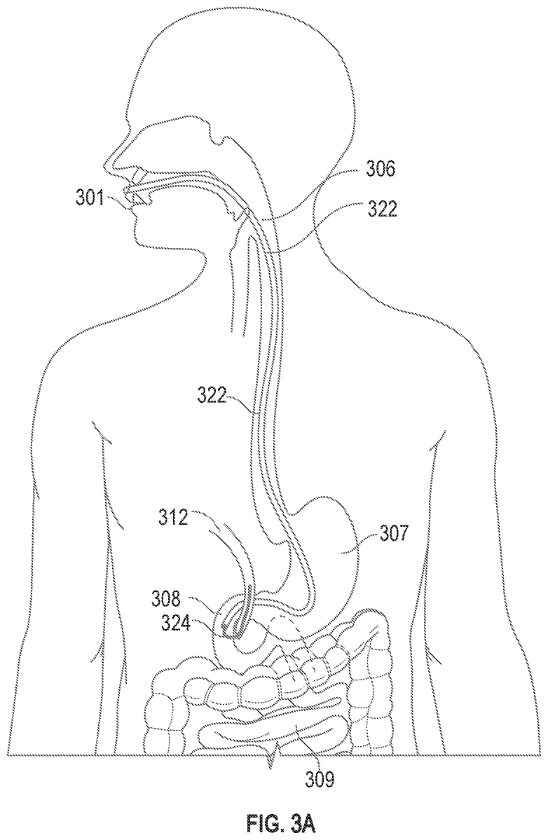

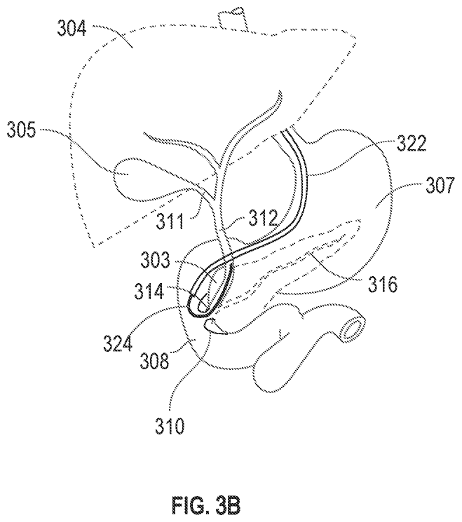

FIGS. 3A-3B are diagrams illustrating an example of peroral cholangioscopy performed via direct insertion of a cholangioscope 324 into the bile duct, as in a DPOC procedure, and a portion of patient anatomy where the procedure is performed. The cholangioscope 324 is nested inside of a guide sheath 322, and inserted perorally into a patient to reach duodenum 308. Duodenum 308 comprises an upper part of the small intestine. The guide sheath 322 can extend into mouth 301, through esophagus 306, through stomach 307 to reach the duodenum 308. Before reaching intestines 309, the guide sheath 322 can position the cholangioscope 324 proximate common bile duct 312. The common bile duct 312 carries bile from the gallbladder 305 and liver 304, and empties the bile into the duodenum 308 through sphincter of Oddi 310 (FIG. 3B). The cholangioscope 324 can extend from guide sheath 322 to extend into common bile duct 312. In some examples, steering features of guide sheath 322 (e.g., pull wire) can be used to facilitate navigating and bending of cholangioscope 324 through stomach 307, in addition to direct steering of cholangioscope 324 via the pull wires. For example, navigation of the Pyloric canal and Pyloric sphincter can be difficult to navigate using only an endoscope. Thus, the guide sheath 322 can be used to turn or bend elongate body of cholangioscope 324, or reduce the amount of steering or bending of the elongate body of the cholangioscope 324 required by pull wires, to facilitate traversing the Pyloric sphincter.

FIG. 3B is a schematic view of duodenum 308 connected to common bile duct 312 via duodenal papilla 314. Common bile duct 312 can branch off into pancreatic duct 316 and gallbladder duct 311. Duodenal papilla 314 can include sphincter of Oddi 310 that controls flow of bile and pancreatic juice into the intestine (duodenum). Pancreatic duct 316 can lead to pancreas 303. Pancreatic duct 316 carries pancreatic juice from pancreas 303 to the common bile duct 312. Gallbladder duct 311 can lead to gallbladder 305. In some patients, it can be difficult to navigate surgical instruments to duodenal papilla 314. It can also be difficult to navigate a surgical instrument into common bile duct 312 via insertion through duodenal papilla 314. Therefore, it is common during medical procedures to cut sphincter of Oddi 310 to enlarge duodenal papilla 314 to allow for easier access of instrument into common bile duct 312.

FIG. 4 is a diagram illustrating an example of mother-daughter endoscopes used in an ERCP procedure, and a portion of patient anatomy where the procedure is performed. The mother-daughter endoscopes comprise an auxiliary scope 434 (cholangioscope) attached to and advanced through a lumen 432 of a main scope 400 (duodenoscope). The auxiliary scope 434 can comprise a lumen 436. The distal portion of the main scope 400 positioned in duodenum 308 comprises a functional module 402, an insertion section module 404, and a control module 406. The control module 406 can include, or be coupled to, a controller 408. Similar to the discussion above with respect to FIG. 1, the control module 406 can include other components, such as those described with reference to endoscopy system 10 (FIG. 1) and control unit 16 (FIG. 2). Additionally, the control module 406 can comprise components for controlling an imaging device (e.g., a camera) and a light source connected to the auxiliary scope 434, such as an imaging unit 410, a lighting unit 412 and a power unit 414. The main scope 400 can be configured similarly as endoscope 14 of FIGS. 1 and 2.

The functional module 402 of the main scope 400 can comprise an elevator portion 430. The auxiliary scope 434 can itself include functional components, such as camera lens 437 and a light lens (not illustrated) coupled to control module 406, to facilitate navigation of the auxiliary scope 434 from the main scope 400 through the anatomy and to facilitate viewing of components extending from lumen 432.

In ERCP, the auxiliary scope 434 can be guided into the sphincter of Oddi 310. Therefrom, a surgeon operating the auxiliary scope 434 can navigate the auxiliary scope 434 through the lumen 432 of the main scope toward the gallbladder 305, liver 304, or other locations in the gastrointestinal system to perform various procedures. In some examples, the auxiliary scope 434 can be used to guide an additional device to the anatomy to obtain biological matter (e.g., tissue), such as by passage through or attachment to lumen 436.

The biological sample matter can be removed from the patient, typically by removal of the additional device from the auxiliary device, so that the removed biological matter can be analyzed to diagnose one or more conditions of the patient. According to several examples, the mother-daughter endoscope assembly (including the main scope 400 and the auxiliary scope 434) can include additional device features, such as forceps or an auger, for gathering and removing cancerous or pre-cancerous matter (e.g., carcinoma, sarcoma, myeloma, leukemia, lymphoma and the like), or performing endometriosis evaluation, biliary ductal biopsies, and the like.

The controller 408 can include, or be coupled to, a treatment plan generator 460. The treatment plan generator 460, which is an example of the treatment generator 44 as illustrated in FIG. 2, can automatically generate a pancreaticobiliary access plan for passing at least a portion of the endoscope 14 (or other steerable elongate instrument such as a catheter or a guidewire) into the pancreaticobiliary system to perform a diagnostic or therapeutic operation therein.

The treatment plan generator 460 can generate alternative pancreaticobiliary access approaches that are easier to perform than conventional approaches such as direct cutting (e.g., sphincterotomy). One of such alternative approaches, also referred to as an RF-based approach, involves delivering RF energy to the stricture via a working head of the endoscope (or other steerable elongate instrument such as a catheter). Another alternative approach, also referred to as a mechanical puncture-based approach, involves applying mechanical force to the stricture via a working head of sufficient stiffness at a distal portion of the endoscope (or other steerable elongate instrument such as a catheter or a guidewire). The treatment plan generator 460 can include an AI-based access decision system 462 that can identify an entry site at or around a stricture, and to determine a proper pancreaticobiliary access method for the patient, such as between the RF-based approach and the mechanical puncture-based approach, based at least on images of the patient anatomy of interest. Images of the stricture and neighboring environment can be obtained from imaging studies, such as endoscopic images, X-ray images, fluoroscopy images, CT images, MRI images such as image obtained from Magnetic resonance cholangiopancreatography (MRCP), or endoscopic ultrasonography (EUS) images.

In an example, RF energy or mechanical force can be applied to an entry site of a stricture adjacent to duodenal papilla to gain pancreaticobiliary access therefrom. The AI-based access decision system 462 can use images of duodenal papilla (e.g., endoscopic images captured by an imaging device (e.g., a camera) of the endoscope, or images produced by other external imaging devices) to identify patient candidacy for the RF-based approach, or to recommend between the RF-based approach and the mechanical puncture-based approach.

The AI-based access decision system 462 can include an image processing unit 463 and at least one trained machine-learning (ML) model 464. The image processing unit 463 can receive images of strictures at duodenal papilla and its surrounding environment acquired during an endoscopic procedure, and extract one or more geometric or morphological features from the image. The images or image features extracted therefrom can be applied to the at least one trained ML model 464 to automatically determine patient candidacy for the RF-based approach.

The at least one trained ML model 464 can have a neural network structure comprising an input layer, one or more hidden layers, and an output layer. To train the ML model, images or image features produced by the image processing unit 463, optionally along with other input data (e.g., sensor signals indicative of anatomical characteristics of the stricture, patient general health status, etc.), can be fed into the input layer of the ML model, which propagates the input data or data features through one or more hidden layers to the output layer. The trained ML model 464 can provide the AI-based access decision system 462 with the ability to perform tasks, without explicitly being programmed, by making inferences based on patterns found in the analysis of data. The trained ML model 464 explores the study and construction of algorithms (e.g., ML algorithms) that may learn from existing data and make predictions about new data. Such algorithms operate by building the trained ML model 464 from training data in order to make data-driven predictions or decisions expressed as outputs or assessments.

The ML model may be trained using supervised learning or unsupervised learning. Supervised learning uses prior knowledge (e.g., examples that correlate inputs to outputs or outcomes) to learn the relationships between the inputs and the outputs. The goal of supervised learning is to learn a function that, given some training data, best approximates the relationship between the training inputs and outputs so that the ML model can implement the same relationships when given inputs to generate the corresponding outputs. Unsupervised learning is the training of an ML algorithm using information that is neither classified nor labeled, and allowing the algorithm to act on that information without guidance. Unsupervised learning is useful in exploratory analysis because it can automatically identify structure in data.

Common tasks for supervised learning are classification problems and regression problems. Classification problems, also referred to as categorization problems, aim at classifying items into one of several category values. Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM). Examples of DNN include a convolutional neural network (CNN), a recurrent neural network (RNN), a deep belief network (DBN), or a hybrid neural network comprising two or more neural network models of different types or different model configurations.

Some common tasks for unsupervised learning include clustering, representation learning, and density estimation. Some examples of commonly used unsupervised learning algorithms are K-means clustering, principal component analysis, and autoencoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

The at least one trained ML model 464 may be trained using a training module included in the AI-based access decision system 462. Alternatively, the training module can be implemented in a separate unit. To train a ML model, a training dataset can be constructed using past endoscopic procedure data. The past endoscopic procedure data can be stored in a database accessible by the AI-based access decision system 462. The training data may include stored pancreaticobiliary access data obtained from past endoscopic stricture management procedures involving RF-based or mechanical puncture-based approaches to access pancreaticobiliary system on a plurality of patients. Examples of the stored past procedure data can include images of strictures at or around duodenal papilla obtained from the plurality of patients. The training data may additionally include pancreaticobiliary access approaches used in previous procedures (RF-based approach or the mechanical puncture-based approach), navigation parameters associated with the procedure (e.g., position, heading direction or angle, amount of protrusion, speed or force applied to the endoscope, or navigation path toward the stricture, among others), and assessment of outcomes of the procedures (e.g., success rate and patient complications).

In an example, the training data can be screened such that only data of procedures performed by certain physicians (such as those with substantially similar experience levels to the operating physician), and/or data of procedures on certain patients with special requirement (such as those with substantially similar anatomy or patient medical information to the present patient) are included in the training dataset. In an example, the training data can be screened based on a success rate of the procedure, including times of attempts before a successful cannulation or navigation, such that only data of procedures with a desirable success rate achieved within a specified number of attempts are included in the training dataset. In another example, the training data can be screened based on complication associated with the patients. In some examples, particularly in case of a small training dataset (such as due to data screening), the ML model can be trained to identify an entry site at or around a pancreaticobiliary stricture, and to determine a pancreaticobiliary access approach such as the RF-based approach or the mechanical puncture-based approach by extrapolating, interpolating, or bootstrapping the training data. The training of the ML model may be performed continuously or periodically, or in near real time as additional procedure data are made available. The training involves algorithmically adjusting one or more ML model parameters, until the ML model being trained satisfies a specified training convergence criterion.

The trained ML model can be validated, and implemented in the AI-based access decision system 462. The AI-based access decision system 462 may apply an image of the stricture and the surrounding environment (or the image features such as generated by the image processing unit 463), to the at least one trained ML model 464 to identify an entry site at or around the stricture, and to determine patient candidacy for the RF-based approach to gain pancreaticobiliary access. In some examples, a first ML model can be trained to identify an entry site at or around the stricture, and a different second ML model can be trained to determine patient candidacy for the RF-based approach to gain pancreaticobiliary access. In some examples, the AI-based access decision system 462 may generate a recommendation to the user of either RF-based approach or mechanical puncture-based approach for use in the procedure. In some examples, for the identified candidate suitable to be treated with the RF-based approach, the treatment plan generator 460 may identify characteristics of the strictures, such as by using images of the stricture or sensor data acquired by sensors associated with the RF catheter 540. The controller 408 may adjust output of an RF power generator based at least on the identified characteristics of the stricture.

FIGS. 5A-5B are diagrams illustrating examples alternative endoscopic approaches to gain access to the pancreaticobiliary system (e.g., the common bile duct or the pancreatic duct) in the presence of strictures. Unlike conventional sphincterotomy which requires cutting of the biliary sphincter and intraduodenal segment of the common bile duct following selective cannulation, alternative pancreaticobiliary access approaches as illustrated in FIGS. 5A-5B use an RF catheter or a specialized puncture device (e.g., wire) to open up the stricture and gain access to the pancreaticobiliary region where diagnostic or therapeutic interventions can be performed.

FIG. 5A illustrates an example of a mechanical puncture-based approach for accessing a pancreaticobiliary region in a ERCP procedure. A puncture catheter 520 can be advanced alongside or via a channel of a flexible endoscope 500 (or other steerable elongate instrument such as a guidewire, a catheter, or a guide sheath), pass through the GI tract and exit to duodenal papilla 511. Pancreaticobiliary stricture tissue 513 in the common bile duct or strictures at or around the duodenal papilla 511 may cause narrowing or obstruction that prevents direct access to the pancreaticobiliary system (e.g., common bile duct). The puncture catheter 520 can be extended from the endoscope 500 and advanced toward a target entry site at or around the pancreaticobiliary stricture tissue 513, such as a portion of the duodenal papilla 511 or beside ampulla of Vater 512. In an example, the entry site may be identified using an ML model, as discussed above with reference to FIG. 4. A working head 522, located at a distal portion of the puncture catheter 520, can be positioned at the entry site. Mechanical force exerted manually (e.g., by the operating physician) or caused by a robotic apparatus (e.g., a robot arm), can be applied to the working head 522 to produce an opening sized to pass at least the distal portion of the puncture catheter 520 or other tools or tool delivery system into the pancreaticobiliary system (e.g., the common bile duct).

The working head 522 can be made of material having a higher amount of stiffness than the proximal portion of the puncture catheter 520. The stiff working head can facilitate puncturing a hole at the entry site at or around the stricture without bending. The more compliant proximal portion can promote flexible movement of the puncture catheter 520 inside the working channel of the endoscope 500. In an example, the distal portion of the puncture catheter 520 (including the working head 522) can be made of material through a rigidization process. In an example, the distal portion including the working head 522 can be configured to have axially variable stiffness, such that the working head 522 can achieve a higher amount of stiffness as the working head approaches the stricture. In an example, at least the distal portion of the puncture catheter 520 comprises struts spatially arranged to provide variable stiffness as the distal portion of the puncture catheter 520 changes its posture. For example, when the distal portion of the puncture catheter 520 changes from a bending posture to a straightening posture, the structs change their spatially arrangement to provide an increased stiffness at the working head 522. Such catheter posture-dependent stiffness allows for flexible motion of the puncture catheter 520 inside the working channel of the endoscope 500 and controllable and efficient puncturing at the entry site simply by straightening the working head 522.

FIG. 5B illustrates an example of an RF-based approach for accessing a pancreaticobiliary region in a ERCP procedure. During ERCP, an RF catheter 540, coupled to an RF power generator, can be advanced alongside or via a channel of a flexible endoscope 500 (or other steerable elongate instrument such as a guidewire, a catheter, or a guide sheath), pass through the GI tract and exit to duodenal papilla 511. In an example, the pancreaticobiliary stricture may present at or around the duodenal papilla 511 causing narrowing or obstruction therein that prevents direct access to the pancreaticobiliary system (e.g., common bile duct) through duodenal papilla 511, as shown in FIG. 5A. In another example, as shown in FIG. 5B, pancreaticobiliary stricture tissue 533 may present at the ampulla of Vater or proximal portion of the common bile duct. The RF catheter 540 can be extended from the endoscope 500 towards a target entry site at or around the pancreaticobiliary stricture tissue 533 beside ampulla of Vater 512 or near the duodenal papilla 511. In an example, the entry site may be identified using an ML model, as discussed above. RF energy, generated by the RF power generator, can be applied to electrodes disposed on a working head 542 at a distal portion of the RF catheter 540. The working head 542 may be made of uncoiled metal wire of sufficient rigidity and geometric dimensions (e.g., small diameter) to facilitate flexible movement in the pancreaticobiliary anatomy and through the RF-treated stricture. The RF energy can ablate the tissue of contact to create a passageway for passing at least the distal portion of the RF catheter 540 or other tools or tool delivery system into a pancreaticobiliary region of interest, where therapeutic or diagnostic operations can be performed. In some examples, output of the RF power generator may be adjusted automatically based on the characteristics of the strictures, such as generated by the treatment plan generator 460 using images of the stricture or sensor data acquired by sensors associated with the RF catheter 540, as described above with reference to FIG. 4.

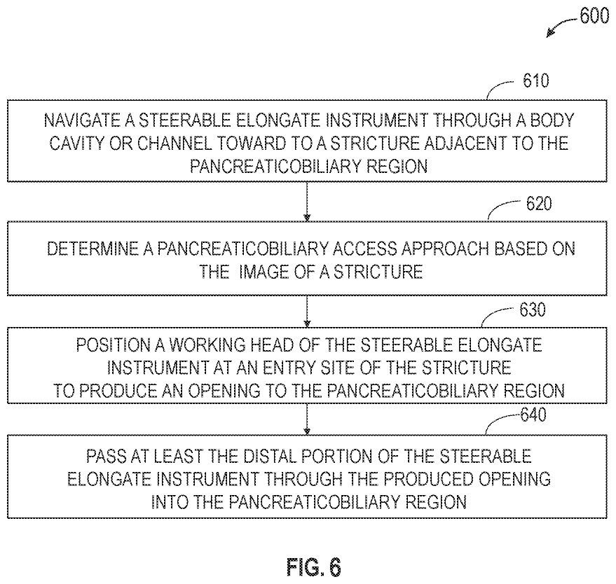

FIG. 6 is a flow chart illustrating an example method 600 for accessing a pancreaticobiliary region of a patient to perform diagnostic or therapeutic operations therein. The pancreaticobiliary access can be achieved using either a mechanical puncture-based approach such as that described above with reference to FIG. 5A, or a radio frequency (RF)-based approach such as that described above with reference to FIG. 5B.

At 610, a steerable elongate instrument can be navigated through a body cavity or channel, such as portion of patient GI tract including the mouth, the esophagus, the stomach, and the duodenum, as illustrated in FIGS. 5A-5B. Examples of the steerable elongate instrument may include an elongate portion of an flexible endoscope, a guidewire, a catheter, or a guide sheath. The steerable elongate instrument can be advanced toward to a stricture adjacent to a pancreaticobiliary region, such as a stricture at or around the duodenal papilla as shown in FIG. 5A, or a stricture at the ampulla of Vater or proximal portion of the common bile duct as shown in FIG. 5B.

At 620, a pancreaticobiliary access approach can be determined, such as between an RF-based approach and a mechanical puncture-based approach. The RF-based approach involves applying RF energy, provided by a RF power generator, to an entry site at or around the stricture via a working head of the steerable elongate instrument to produce an opening to the pancreaticobiliary region. The mechanical puncture-based approach involves applying mechanical force to a working head of the steerable elongate instrument positioned at the entry site at or around the stricture to create an opening sized to pass at least the distal portion of the steerable elongate instrument therethrough and into the pancreaticobiliary region.

The entry site can be identified automatically based at least on images of the stricture and neighboring environment, which can be obtained from imaging studies before or during the procedure. Examples of the images can include endoscopic images (e.g., image from cholangioscopy), X-ray images, fluoroscopy images, CT images, MRI images such as image obtained from Magnetic resonance cholangiopancreatography (MRCP), or endoscopic ultrasonography (EUS) images. In an example, the entry site can be identified using artificial intelligence (AI) or machine learning (ML), such as by the AI-based access decision system 462 as described above with reference to FIG. 4.

In an example, a ML model can be trained using a training dataset including stored pancreaticobiliary access data obtained from past endoscopic stricture management procedures involving RF-based or mechanical puncture-based pancreaticobiliary access approaches on a plurality of patients. Examples of the stored past procedure data can include images of strictures at or around duodenal papilla obtained from the plurality of patients. The training data may additionally include pancreaticobiliary access approaches used in previous procedures (RF-based approach or the mechanical puncture-based approach), navigation parameters associated with the procedure (e.g., position, heading direction or angle, amount of protrusion, speed or force applied to the endoscope, or navigation path toward the stricture, among others), and assessment of outcomes of the procedures (e.g., success rate and patient complications). The trained ML model can be used to identify an entry site at or around the stricture to receive RF energy or puncture force, and to identify patient candidacy for RF-based approach. For non-candidates, mechanical puncture-based approach may be recommended.

At 630, a working head of the steerable elongate instrument can be positioned at the identified entry site of a stricture adjacent to a pancreaticobiliary region to produce an opening to the pancreaticobiliary region in accordance with pancreaticobiliary access approach determined at 620. If an RF-based approach is selected at 620, then RF energy can be delivered to the entry cite of the stricture via the working head of the steerable elongate instrument (e.g., the RF catheter 540 as shown in FIG. 5B). In an example, the working head may be made of uncoiled metal wire of sufficient rigidity and geometric dimensions (e.g., small diameter) to facilitate flexible movement in the pancreaticobiliary anatomy and through the RF-treated stricture. The RF energy can ablate the tissue of contact to create a passageway for passing at least the distal portion of the steerable elongate instrument or other tools or tool delivery system into pancreaticobiliary region of interest. In some examples, output of the RF power generator may be adjusted automatically based on the characteristics of the strictures.

If a mechanical puncture-based approach is selected at 620, then mechanical force may be applied to the working head of the steerable elongate instrument (e.g., the puncture catheter 520 as shown in FIG. 5A). Mechanical force can by exerted manually (e.g., by the operating physician), or caused by a robotic apparatus (e.g., a robot arm). An opening can be created at the entry site of the stricture sized to pass at least the distal portion of the steerable elongate instrument or other tools or tool delivery system into pancreaticobiliary region of interest. In some examples, the working head can be made of material having a higher amount of stiffness than the proximal portion of the puncture catheter. The stiff working head can facilitate puncturing a hole at the entry site at or around the stricture without bending. The more compliant proximal portion can promote flexible movement of the puncture catheter inside the working channel of the endoscope. In an example, the working head can be made of material through a rigidization process. In an example, the distal portion (including the working head) can be configured to have axially variable stiffness, such that the working head can achieve a higher amount of stiffness as the working head approaches the stricture. In an example, at least the distal portion of the puncture catheter comprises struts spatially arranged to provide variable stiffness as the distal portion of the puncture catheter changes its posture. Such catheter posture-dependent stiffness allows for flexible motion of the puncture catheter inside the working channel of the endoscope and controllable and efficient puncturing at the entry site simply by straightening the working head.

At 640, at least a distal portion of the steerable elongate instrument can be passed through the opening created at 630 (either via the RF-based approach or the mechanical puncture-based approach), and into the pancreaticobiliary region, where a diagnostic or therapeutic operation can be performed.

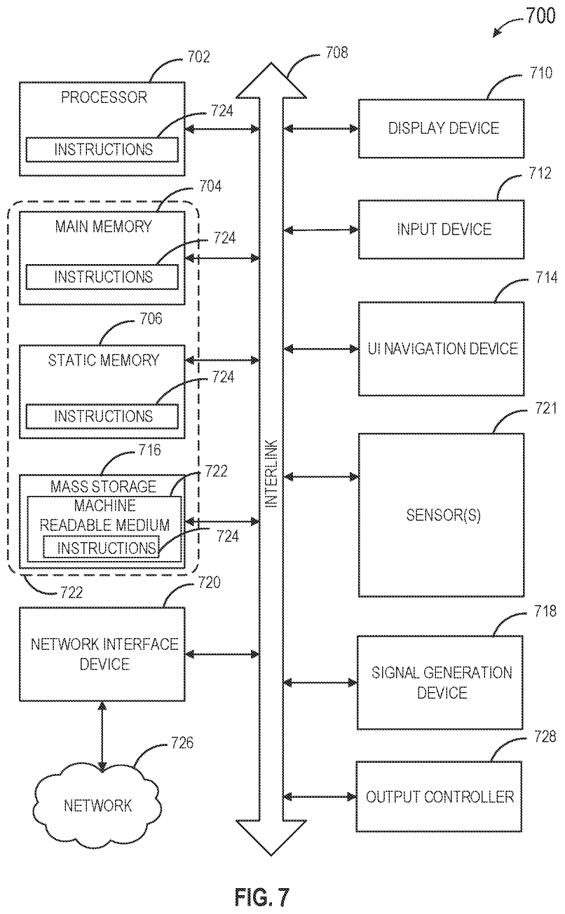

FIG. 7 illustrates generally a block diagram of an example machine 700 upon which any one or more of the techniques (e.g., methodologies) discussed herein may perform. Portions of this description may apply to the computing framework of various portions of the treatment plan generator 460, such as the AI-based access decision system 462.

In alternative embodiments, the machine 700 may operate as a standalone device or may be connected (e.g., networked) to other machines. In a networked deployment, the machine 700 may operate in the capacity of a server machine, a client machine, or both in server-client network environments. In an example, the machine 700 may act as a peer machine in peer-to-peer (P2P) (or other distributed) network environment. The machine 700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 700 may include a hardware processor 702 (e.g., a central processing unit (CPU), a graphics processing unit (GPU), a hardware processor core, or any combination thereof), a main memory 704 and a static memory 706, some or all of which may communicate with each other via an interlink (e.g., bus) 708. The machine 700 may further include a display unit 710 (e.g., a raster display, vector display, holographic display, etc.), an alphanumeric input device 712 (e.g., a keyboard), and a user interface (UI) navigation device 714 (e.g., a mouse). In an example, the display unit 710, input device 712 and UI navigation device 714 may be a touch screen display. The machine 700 may additionally include a storage device (e.g., drive unit) 716, a signal generation device 718 (e.g., a speaker), a network interface device 720, and one or more sensors 721, such as a global positioning system (GPS) sensor, compass, accelerometer, or other sensors. The machine 700 may include an output controller 728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 716 may include a machine readable medium 722 on which is stored one or more sets of data structures or instructions 724 (e.g., software) embodying or utilized by any one or more of the techniques or functions described herein. The instructions 724 may also reside, completely or at least partially, within the main memory 704, within static memory 706, or within the hardware processor 702 during execution thereof by the machine 700. In an example, one or any combination of the hardware processor 702, the main memory 704, the static memory 706, or the storage device 716 may constitute machine readable media.

While the machine-readable medium 722 is illustrated as a single medium, the term “machine readable medium” may include a single medium or multiple media (e.g., a centralized or distributed database, and/or associated caches and servers) configured to store the one or more instructions 724.

The term “machine readable medium” may include any medium that is capable of storing, encoding, or carrying instructions for execution by the machine 700 and that cause the machine 700 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 724 may further be transmitted or received over a communication network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 720 may include one or more physical jacks (e.g., Ethernet, coaxial, or phone jacks) or one or more antennas to connect to the communication network 726. In an example, the network interface device 720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 700, and includes digital or analog communications signals or other intangible medium to facilitate communication of such software.

Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.
1. A method for endoscopically accessing a pancreaticobiliary region of a patient, the method comprising:
   navigating a steerable elongate instrument through a body cavity or channel toward a stricture adjacent to the pancreaticobiliary region;
   delivering radio-frequency (RF) energy to an entry site of the stricture via a working head of the steerable elongate instrument to produce an opening to the pancreaticobiliary region; and
   passing at least a distal portion of the steerable elongate instrument through the produced opening into the pancreaticobiliary region to perform a diagnostic or therapeutic operation therein.
2. The method of example 1, further comprising applying an image of the stricture to a trained machine-learning (ML) model to identify the entry site of the stricture.
3. The method of any of examples 1-2, wherein the RF energy is applied to a stricture beside an ampulla of Vater to produce an opening to a common bile duct.
4. The method of any of examples 1-3, wherein delivering the RF energy is through an uncoiled wire portion on the working head of the steerable elongate instrument, the uncoiled wire portion electrically coupled to an RF power generator.
5. The method of any of examples 1-4, comprising adjusting an RF energy delivered to the entry site of the stricture based at least on a characteristic of the stricture.
6. A method for accessing a pancreaticobiliary region of a patient, the method comprising:
   navigating a steerable elongate instrument through a body cavity or channel toward to a stricture adjacent to the pancreaticobiliary region, the steerable elongate instrument extended between a proximal portion and a distal portion, the distal portion including a working head configured to achieve a higher amount of stiffness than the proximal portion of the steerable elongate instrument as the working head approaches the stricture;
   positioning the working head of the steerable elongate instrument at an entry site of the stricture and applying a mechanical force thereto to produce an opening to the pancreaticobiliary region; and
   passing at least the distal portion of the steerable elongate instrument through the produced opening into the pancreaticobiliary region to perform diagnostic or therapeutic operation therein.
7. The method of examples 6, further comprising applying an image of the stricture to a trained machine-learning (ML) model to identify the entry site of the stricture.
8. The method of any of examples 6-7, wherein the working head is made of material through a rigidization process.
9. The method of any of examples 6-8, wherein the distal portion of the steerable elongate instrument is configured to have axially variable stiffness.
10. The method of any of examples 6-9, wherein the distal portion of the steerable elongate instrument comprises struts spatially arranged to provide variable stiffness as the steerable elongate instrument changes its posture, including an increase in stiffness in response to a change from a bending posture to a straightening posture.
11. An endoscopic system, comprising:
   a steerable elongate instrument extended between a proximal portion and a distal portion, the distal portion including a working head configured to achieve a higher amount stiffness than other portions of the steerable elongate instrument as the working head approaches a stricture adjacent to a pancreaticobiliary region; and
   a controller configured to provide a control signal to an actuator to robotically facilitate navigation and manipulation of the steerable elongate instrument, including, via the actuator:
   position the working head of the steerable elongate instrument at an entry site of the stricture and apply a mechanical force thereto to produce an opening to the pancreaticobiliary region; and
   pass at least the distal portion of the steerable elongate instrument through the produced opening into the pancreaticobiliary region to perform a diagnostic or therapeutic operation therein.
12. The endoscopic system of example 11, comprising a robot arm configured to detachably engage the steerable elongate instrument, and to automatically adjust position or navigation of the steerable elongate instrument via the actuator in response to the control signal.
13. The endoscopic system of any of examples 11-12, wherein the steerable elongate instrument is configured to be robotically positioned and navigated to a duodenal papilla or a portion of pancreaticobiliary system.
14. The endoscopic system of any of examples 11-13, wherein the working head is made of material through a rigidization process.
15. An endoscopic system, comprising:
   a steerable elongate instrument configured to be positioned and navigated in a patient anatomy;
   a controller configured to:
   receive an image of a stricture adjacent to a pancreaticobiliary region; and
   apply the received image of the stricture to at least one trained machine-learning (ML) model to identify an entry site of the stricture, and to determine a pancreaticobiliary access approach, between (i) an radio frequency (RF)-based approach and (ii) a mechanical puncture-based approach, to access the pancreaticobiliary region; and an output unit configured to provide the determined pancreaticobiliary access approach to a user.
16. The endoscopic system of example 15, wherein the controller is further configured to:
   construct a training dataset comprising stored procedure data obtained from past endoscopic stricture management procedures on a plurality of patients using respective pancreaticobiliary access approaches including the RF-based approach or the mechanical puncture-based approach, the stored procedure data including (i) images of strictures of the plurality of patients and (ii) assessments of the pancreaticobiliary access approaches of the respective procedures; and
   train the ML model using the training dataset.
17. The endoscopic system of any of examples 15-16, wherein the steerable elongate instrument includes a catheter, a guide wire, or a guide sheath including a lumen to pass a stricture management device therethrough.
18. The endoscopic system of any of examples 15-17, wherein the steerable elongate instrument includes an endoscope, the endoscope including an imaging sensor to generate the image of the stricture.
19. The endoscopic system of any of examples 15-18, wherein the steerable elongate instrument is extended between a proximal portion and a distal portion, the distal portion including a working head having a higher amount of stiffness than other portions of the steerable elongate instrument,
   wherein the working head is configured to, in response to a puncture force applied thereto, puncture the entry site of the stricture to produce an opening sized to pass at least the distal portion of the steerable elongate instrument therethrough.
20. The endoscopic system of claim any of examples 15-19, wherein the steerable elongate instrument includes, at a distal portion thereof, a working head configured to be electrically coupled to an RF power generator and to deliver RF energy to the entry site of the stricture to produce an opening sized to pass at least the distal portion of the steerable elongate instrument therethrough.

What is claimed is:

1. A method for endoscopically accessing a pancreaticobiliary region of a patient, the method comprising:
navigating a steerable elongate instrument through a body cavity or channel toward a stricture adjacent to the pancreaticobiliary region;
delivering radio-frequency (RF) energy to an entry site of the stricture via a working head of the steerable elongate instrument to produce an opening to the pancreaticobiliary region; and
passing at least a distal portion of the steerable elongate instrument through the produced opening into the pancreaticobiliary region to perform a diagnostic or therapeutic operation therein.

2. The method of claim 1, further comprising applying an image of the stricture to a trained machine-learning (ML) model to identify the entry site of the stricture.

3. The method of claim 1, wherein the RF energy is applied to a stricture beside an ampulla of Vater to produce an opening to a common bile duct.

4. The method of claim 1, wherein delivering the RF energy is through an uncoiled wire portion on the working head of the steerable elongate instrument, the uncoiled wire portion electrically coupled to an RF power generator.

5. The method of claim 1, comprising adjusting an RF energy delivered to the entry site of the stricture based at least on a characteristic of the stricture.

6. A method for accessing a pancreaticobiliary region of a patient, the method comprising:
navigating a steerable elongate instrument through a body cavity or channel toward to a stricture adjacent to the pancreaticobiliary region, the steerable elongate instrument extended between a proximal portion and a distal portion, the distal portion including a working head configured to achieve a higher amount of stiffness than the proximal portion of the steerable elongate instrument as the working head approaches the stricture:
positioning the working head of the steerable elongate instrument at an entry site of the stricture and applying a mechanical force thereto to produce an opening to the pancreaticobiliary region; and
passing at least the distal portion of the steerable elongate instrument through the produced opening into the pancreaticobiliary region to perform diagnostic or therapeutic operation therein.

7. The method of claim 6, further comprising applying an image of the stricture to a trained machine-learning (ML) model to identify the entry site of the stricture.

8. The method of claim 6, wherein the working head is made of material through a rigidization process.

9. The method of claim 6, wherein the distal portion of the steerable elongate instrument is configured to have axially variable stiffness.

10. The method of claim 6, wherein the distal portion of the steerable elongate instrument comprises struts spatially arranged to provide variable stiffness as the steerable elongate instrument changes its posture, including an increase in stiffness in response to a change from a bending posture to a straightening posture.

15. An endoscopic system, comprising:
a steerable elongate instrument configured to be positioned and navigated in a patient anatomy;
a controller configured to:
receive an image of a stricture adjacent to a pancreaticobiliary region; and
apply the received image of the stricture to at least one trained machine-learning (ML) model to identify an entry site of the stricture, and to determine a pancreaticobiliary access approach, between (i) an radio frequency (RF)-based approach and (ii) a mechanical puncture-based approach, to access the pancreaticobiliary region; and
an output unit configured to provide the determined pancreaticobiliary access approach to a user.

16. The endoscopic system of claim 15, wherein the controller is further configured to:
construct a training dataset comprising stored procedure data obtained from past endoscopic stricture management procedures on a plurality of patients using respective pancreaticobiliary access approaches including the RF-based approach or the mechanical puncture-based approach, the stored procedure data including (i) images of strictures of the plurality of patients and (ii) assessments of the pancreaticobiliary access approaches of the respective procedures; and
train the ML model using the training dataset.

17. The endoscopic system of claim 15, wherein the steerable elongate instrument includes a catheter, a guide wire, or a guide sheath including a lumen to pass a stricture management device therethrough.

18. The endoscopic system of claim 15, wherein the steerable elongate instrument includes an endoscope, the endoscope including an imaging sensor to generate the image of the stricture.

19. The endoscopic system of claim 15, wherein the steerable elongate instrument is extended between a proximal portion and a distal portion, the distal portion including a working head having a higher amount of stiffness than other portions of the steerable elongate instrument,
wherein the working head is configured to, in response to a puncture force applied thereto, puncture the entry site of the stricture to produce an opening sized to pass at least the distal portion of the steerable elongate instrument therethrough.

20. The endoscopic system of claim 15, wherein the steerable elongate instrument includes, at a distal portion thereof, a working head configured to be electrically coupled to an RF power generator and to deliver RF energy to the entry site of the stricture to produce an opening sized to pass at least the distal portion of the steerable elongate instrument therethrough.
